(19) United States
(12) Patent Application Publication
(10) Pub. No.: US 2008/0312290 A1
(43) Pub. Date: Dec. 18, 2008

(54) Oil Based Suspension Concentrates

(75) Inventors: Ronald Vermeer, Leverkusen (DE); Manuela Eberhard, Leverkusen (DE)

Correspondence Address: STERNE, KESSLER, GOLDSTEIN & FOX P.L.L.C., 1100 NEW YORK AVENUE, N.W., WASHINGTON, DC, 20005, US

(73) Assignee: Bayer CropScience AG, Monheim (DE)

(21) Appl. No.: 12/089,845

(22) PCT Filed: Sep. 28, 2006
(86) PCT No.: PCT/EP2006/009433

§ 371 Date: Aug. 27, 2008

(30) Foreign Application Priority Data
Oct. 11, 2005 (DE) 102005048539.1

(51) Int. Cl. A01N 53/02; A01N 51/00; A01N 53/06; A61P 7/04; A01N 25/00

(52) U.S. Cl. 514/341; 514/531; 514/520; 514/783

(57) ABSTRACT

Agrochemical formulation comprising at least one room-temperature-solid active substance from the class of the neonicotinoids, at least one room-temperature-solid active substance from the class of the pyrethroids, at least one penetrant, at least one vegetable oil, cyclohexanone, at least one nonionic surfactant and/or at least one anionic surfactant, and one or more additives, process for producing it, and its use for controlling harmful organisms.

The present invention relates to new, oil-based suspension concentrates of active agrochemicals, to a process for producing these formulations and to their use for applying the active substances comprised.

Systemic active agrochemicals, especially systemic insecticides, in order to develop their biological effect, need a formulation which enables the active substances to be taken up into the plant/target organisms. Usually, therefore, systemic active agrochemicals are formulated as an emulsifiable concentrate (EC), soluble liquid (SL) and/or oil-based suspension concentrate (OD). In an EC and SL the active substance is in dissolved form, while in the case of an OD formulation it is a solid. In the latter case the biological action is made possible by the addition of penetrants. Contact actives such as pyrethroids, for example, are formulated preferably as EC, especially when a high initial action is needed. Suspension concentrates (SC) or wettable granules (WG) are technically possible in the majority of cases, but do not display the requisite initial action.

Mixed formulations of systemic and contact insecticides, such as a mixture of imidacloprid with beta-cyfluthrin, for example, are of great interest as an alternative to the organophosphates, which carry high acute toxicity. Mixtures of this kind are appropriate alternatives to the application of organophosphates only if a high initial action is present and if formulations with a high active substance content are present. There are no known EC formulations with high levels both of imidacloprid and of beta-cyfluthrin, since there is no solvent able to dissolve both active substances in appropriate amount. Consequently, only oil-based or water-free suspension concentrates come into consideration.

Numerous water-free suspension concentrates of active agrochemicals have already been disclosed. For instance EP-A 0 789 999 describes formulations of this type which in addition to active substance and oil comprise a mixture of different surfactants—including surfactants which serve as penetrants—and also a hydrophobicized aluminophyllosilicate as thickener. The cited patent describes suitable active substances as being those which have a solubility in oil of less than 5 g/l, preferably less than 1 g/l, in particular less than 0.1 g/l.

From U.S. Pat. No. 6,165,940, moreover, non-aqueous suspension concentrates are already known in which besides active agrochemical, penetrant and surfactant or surfactant mixture there is an organic solvent, suitable solvents of this type including liquid paraffin or vegetable oil esters. That invention describes suspension concentrates composed of solid active substance(s) and organic solvents, the active substance being less than moderately soluble. A solubility of less than 10 g/l, preferably less than 5 g/l, is explicitly stated.

DE-A 10 129 855 describes further oil-based suspension concentrates which comprise active agrochemicals, penetrants and surfactants.

A disadvantage of the aforementioned formulations is that it is not possible to develop a sparingly soluble (less than 10 g/l) active substance in combination with a moderately soluble (10 to 50 g/l at room temperature) active substance in the form of a stable oil-based suspension concentrate without the occurrence of crystal growth after storage. The growth of active substance crystals in a formulation is a considerable disadvantage for the user, since it may result in clogging of the screens of his or her spraying equipment when the product is applied.

An objective of the present invention is to develop stable, storable, oil-based suspension concentrates composed of a sparingly soluble active substance and a moderately soluble active substance which is present in a higher concentration than the solubility limit in the formulation.

New, oil-based suspension concentrates have now been found, comprising
at least one room-temperature-solid active substance from the class of the neonicotinoids,
at least one room-temperature-solid active substance from the class of the pyrethroids,
at least one penetrant,
at least one vegetable oil,
cyclohexanone,
at least one nonionic surfactant and/or at least one anionic surfactant, and
one or more additives from the groups of the emulsifiers, foam inhibitors, preservatives, antioxidants, spreaders, colorants and/or a thickener.

Suitable penetrants in the present context include all those substances which are usually used to enhance the penetration of active agrochemicals into plants. Penetrants are defined in this context by their ability to penetrate from the aqueous spray liquor and/or from the spray coating into the cuticle of the plant and thereby to increase the mobility of active substances in the cuticle. The method described later on and in the literature (Baur et al., 1997, Pesticide Science 51, 131-152) can be used to determine this property.

Additionally it has been found that the oil-based suspension concentrates of the invention can be produced by mixing
at least one room-temperature-solid active substance from the class of the neonicotinoids,
at least one room-temperature-solid active substance from the class of the pyrethroids,
at least one penetrant,
at least one vegetable oil,
cyclohexanone,
at least one nonionic surfactant and/or at least one anionic surfactant, and
one or more additives from the groups of the emulsifiers, foam inhibitors, preservatives, antioxidants, spreaders, colorants and/or a thickener.
   with one another and optionally subsequently grinding the resultant suspension.

Finally it has been found that the oil-based suspension concentrates of the invention are highly suitable for applying the active agrochemicals comprised to plants and/or their habitat.

It is to be considered extremely surprising that the oil-based suspension concentrates of the invention exhibit very good stability, and in particular that no significant crystal growth was observed even after storage at fluctuating temperature. Also unexpected is the fact that they display a markedly better biological activity than the aforementioned formulations most similar in composition. Particularly unexpected is the fact that a very high initial action of the contact substance is found, in spite of the fact that this active substance is present partly as a solid.

Appropriate active substances are insecticides from the class of the neonicotinoids. They are outstandingly suitable for controlling animal pests. Insecticides from the class of the neonicotinoids can be described by formula (II) below

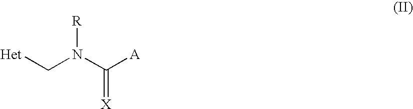

in which
Het is a heterocycle selected from the following group of heterocycles: 2-chloropyrid-5-yl, 2-methylpyrid-5-yl, 1-oxido-3-pyridino, 2-chloro-1-oxido-5-pyridino, 2,3-dichloro-1-oxido-5-pyridino, tetrahydrofuran-3-yl, 5-methyl-tetrahydrofuran-3-yl, 2-chlorothiazol-5-yl,
A is N(R¹)(R²) or S(R²),
   in which
   R¹ is hydrogen, C₁-C₆-alkyl, phenyl-C₁-C₄-alkyl, C₃-C₆-cycloalkyl, C₂-C₆-alkenyl or C₂-C₆-alkynyl and
   R² is C₁-C₆-alkyl, C₂-C₆-alkenyl, C₂-C₆-alkynyl —C(═O)—CH₃ or benzyl,
R is C₁-C₆-alkyl, C₂-C₆-alkenyl, C₂-C₆-alkynyl —C(═O)—CH₃ or benzyl or together with R² is one of the following groups:
   —CH₂—CH₂—, —CH₂—CH₂—CH₂—, —CH₂—O—CH₂—, —CH₂—S—CH₂—, —CH₂—NH—CH₂—, —CH₂—N—(CH₃)—CH₂— and
X is N—NO₂, N—CN or CH—NO₂
   (see for example EP-A1-192 606, EP-A2-580 533, EP-A2-376 279, EP-A2-235 725).

Mention may be made individually of the following compounds which can be used in accordance with the invention.

One compound used with preference in accordance with the invention is thiamethoxam.

Thiamethoxam has the formula

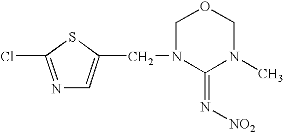

and is known from EP A2 0 580 533.

A further compound used with preference in accordance with the invention is clothianidin.

Clothianidin has the formula

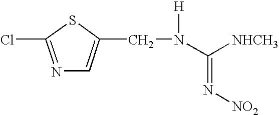

and is known from EP A2 0 376 279.

A further compound used with preference in accordance with the invention is thiacloprid.

Thiacloprid has the formula

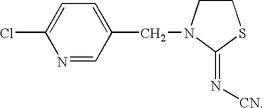

and is known from EP A2 0 235 725.

A further compound used with preference in accordance with the invention is dinotefuran.

Dinotefuran has the formula

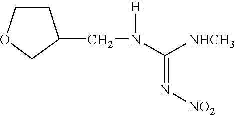

and is known from EP A1 0 649 845.

A further compound used with preference in accordance with the invention is acetamiprid.

Acetamiprid has the formula

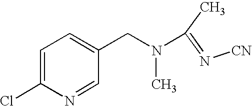

and is known from WO A1 91/04965.

A further compound used with preference in accordance with the invention is nitenpyram.

Nitenpyram has the formula

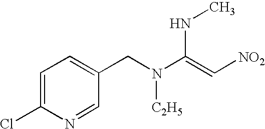

and is known from EP A2 0 302 389.

A further compound used with preference in accordance with the invention is imidacloprid.

Imidacloprid has the formula

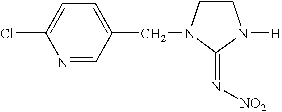

and is known from EP 0 192 060.

Particular preference is given to imidacloprid.

Suitable further active substances include those from the group of the pyrethroids, for example acrinathrin, allethrin (d-cis-trans, d-trans), beta-cyfluthrin, bifenthrin, bioallethrin, bioallethrin-S-cyclopentyl isomer, bioethanomethrin, biopermethrin, bioresmethrin, chlovaporthrin, cis-cypermethrin, cis-resmethrin, cis-permethrin, clocythrin, cycloprothrin, cyfluthrin, cyhalothrin, cypermethrin (alpha-, beta-, theta-, zeta-), cyphenothrin, deltamethrin, empenthrin (1R-isomer), esfenvalerate, etofenprox, fenfluthrin, fenpropathrin, fenpyrithrin, fenvalerate, flubrocythrinate, flucythrinate, flufenprox, flumethrin, fluvalinate, fubfenprox, gamma-cyhalothrin, imiprothrin, kadethrin, lambda-cyhalothrin, metofluthrin, permethrin (cis-, trans-), phenothrin (1R-transisomer), prallethrin, profluthrin, protrifenbute, pyresmethrin, resmethrin, RU 15525, silafluofen, tau-fluvalinate, tefluthrin, terallethrin, tetramethrin (1R isomer), tralomethrin, transfluthrin, ZXI 8901, pyrethrins (pyrethrum). Preference is given to beta-cyfluthrin and deltamethrin.

Preferred penetrants are alkanol alkoxylates of the formula

R—O—(-AO)ₘR′ (I)

in which
R is linear or branched alkyl having 4 to 20 carbon atoms,
R′ is H, methyl, ethyl, n-propyl, isopropyl, n-butyl, isobutyl, tert-butyl, n-pentyl or n-hexyl,
AO is an ethylene oxide radical, a propylene oxide radical, a butylene oxide radical or mixtures of ethylene oxide and propylene oxide radicals or butylene oxide radicals, and
m is a number from 2 to 30.

One particularly preferred group of penetrants are alkanol alkoxylates of the formula

R—O—(-EO—)ₙ—R′ (I-a)

in which
R is as defined above,
R′ is as defined above,
EO is —CH₂—CH₂—O—, and
n is a number from 2 to 20.

A further particularly preferred group of penetrants are alkanol alkoxylates of the formula

R—O—(-EO—)ₚ—(—PO—)_{q}—R (I-b)

in which
R is as defined above,
R′ is as defined above,
EO is —CH₂—CH₂—O—,
PO is

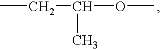

p is a number from 1 to 10, and
q is a number from 1 to 10.

A further particularly preferred group of penetrants are alkanol alkoxylates of the formula

R—O—(—PO—)ᵣ-(EO—)ₛ—R′ (I-c)

in which
R is as defined above,
R′ is as defined above,
EO is —CH₂—CH₂—O—,
PO is

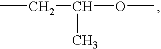

r is a number from 1 to 10, and
s is a number from 1 to 10.

A further particularly preferred group of penetrants are alkanol alkoxylates of the formula (I-e)

R—O(-EO—)ₚ—(—BO)_{q}—R′ (I-d)

in which
R and R′ are as defined above,
EO is CH₂—CH₂—O—,
BO is

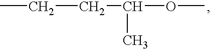

p is a number from 1 to 10, and
q is a number from 1 to 10.

A further particularly preferred group of penetrants are alkanol alkoxylates of the formula (I-f)

R—O—(—BO—)ᵣ—(-EO—)ₛ—R′ (I-e)

in which
R and R′ are as defined above,
BO is

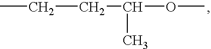

EO is CH₂—CH₂—O—,
r is a number from 1 to 10, and
s is a number from 1 to 10.

A further particularly preferred group of penetrants are alkanol alkoxylates of the formula

CH₃—(CH₂)ₜ—CH₂—O—(—CH₂—CH₂—O—)ᵤ—R′ (I-f)

in which
R′ is as defined above,
t is a number from 8 to 13, and
u is a number from 6 to 17.

In the formulae given above
R is preferably butyl, isobutyl, n-pentyl, isopentyl, neopentyl, n-hexyl, isohexyl, n-octyl, isooctyl, 2-ethylhexyl, nonyl, isononyl, decyl, n-dodecyl, isododecyl, lauryl, myristyl, isotridecyl, trimethylnonyl, palmityl, stearyl or eicosyl.

An example that may be mentioned of an alkanol alkoxylate of the formula (I-c) is 2-ethylhexyl alkoxylate of the formula

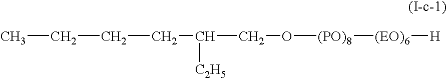

in which
EO is —CH₂—CH₂—O—,
PO is

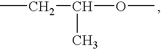

the numbers 8 and 6 represent average values.

An example that may be mentioned of an alkanol alkoxylate of the formula (I-d) is the formula

CH₃—(CH₂)₁₀—O—(-EO—)₆—(—BO—)₂—CH₃ (I-d-1)

in which
EO is CH₂—CH₂—O—,
BO is

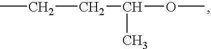

the numbers 10, 6 and 2 represent average values.

Particularly preferred alkanol alkoxylates of the formula (I-f) are compounds of this formula in which
t is a number from 9 to 12 and
u is a number from 7 to 9.

With very particular preference mention may be made of alkanol alkoxylate of the formula (I-f-1)

CH₃—(CH₂)ₜ—CH₂—O—(—CH₂—CH₂—O—)ᵤ—H′ (I-f-1)

in which
t is the average value 10.5 and
u is the average value 8.4.

Mention may likewise be made with very particular preference of alkanol alkoxylate of the formula (I-f-1-1)

CH₃—(CH₂)ₜ—CH₂—O—(—CH₂—CH₂—O—)ᵤ—H (I-f-1-1)

in which
t is the average value 10.5 and
u is the average value 8.4.

A general definition of the alkanol alkoxylates is given by the above formulae. These substances are mixtures of substances of the stated type with different chain lengths. The indices therefore have average values which may also deviate from whole numbers.

The alkanol alkoxylates of the formulae indicated are known or can be prepared by known methods (cf. WO 98-35 553, WO 00-35 278 and EP-A 0 681 865).

Suitable vegetable oils include all oils which can normally be used in agrochemical compositions and can be obtained from plants. Examples that may be mentioned include sunflower oil, rapeseed oil, olive oil, castor oil, colza oil, maize seed oil, cottonseed oil and soybean oil.

The oil-based suspension concentrates of the invention comprise at least one nonionic surfactant or dispersant and/or at least one anionic surfactant or dispersant.

Suitable nonionic surfactants or dispersants include all substances of this type that can normally be used in agrochemical compositions. Preferably mention may be made of polyethylene oxide-polypropylene oxide block copolymers, polyethylene glycol ethers of linear alcohols, reaction products of fatty acids with ethylene oxide and/or propylene oxide, and also polyvinyl alcohol, polyvinylpyrrolidone, copolymers of polyvinyl alcohol and polyvinylpyrrolidone, and copolymers of (meth)acrylic acid and (meth)acrylic esters, and also alkyl ethoxylates and alkylaryl ethoxylates, which optionally may be phosphated and optionally may be utilized with bases, it being possible for mention to be made, by way of example, of sorbitol ethoxylates, and also polyoxyalkylenamine derivatives.

Suitable anionic surfactants include all substances of this type that can normally be used in agrochemical compositions. Preference is given to alkali metal salts and alkaline earth metal salts of alkylsulphonic acids or alkylarylsulphonic acids.

A further preferred group of anionic surfactants or dispersants includes the following salts that are of low solubility in vegetable oil: salts of polystyrenesulphonic acids, salts of polyvinylsulphonic acids, salts of naphthalenesulphonic acid-formaldehyde condensation products, salts of condensation products of naphthalenesulphonic acid, phenolsulphonic acid and formaldehyde, and salts of lignosulphonic acid.

Suitable additives which may be included in the formulations of the invention are emulsifiers, foam inhibitors, preservatives, antioxidants, spreaders, colorants and thickeners.

Preferred emulsifiers are ethoxylated nonylphenols, reaction products of alkylphenols with ethylene oxide and/or propylene oxide, ethoxylated arylalkylphenols, and also ethoxylated and propoxylated arylalkylphenols, and also sulphated or phosphated arylalkyl ethoxylates and/or arylalkyl ethoxy-propoxylates, it being possible to mention, by way of example, sorbitan derivatives, such as polyethylene oxide-sorbitan fatty acid esters and sorbitan fatty acid esters.

Suitable foam inhibitors include all substances that can normally be used for this purpose in agrochemical compositions. Preference is given to silicone oils and magnesium stearate.

Suitable preservatives include all substances that can normally be used for this purpose in agrochemical compositions of this type. Examples that may be mentioned include Preventol® (Bayer AG) and Proxel®.

Suitable antioxidants include all substances that can normally be used for this purpose in agrochemical compositions. Preference is given to butylated hydroxytoluene and/or citric acid.

Suitable spreaders include all substances that can normally be used for this purpose in agrochemical compositions. Preference is given to alkylsiloxanes.

Suitable colorants include all substances that can normally be used for this purpose in agrochemical compositions. Mention may be made, by way of example, of titanium dioxide, pigmentary carbon black, zinc oxide and blue pigments, and also Permanent Red FGR.

Suitable thickeners include all substances that can normally be used for this purpose in agrochemical compositions and which function as thickeners. Preference is given to inorganic particles, such as carbonates, silicates and oxides, and also organic substances, such as urea-formaldehyde condensates. By way of example mention may be made of kaolin, rutile, silicon dioxide, so-called highly disperse silica, silica gels, and also natural and synthetic silicates, and additionally talc.

In one particular embodiment the formulations of the invention may further comprise at least one additional active substance (insecticides, attractants, sterilants, bactericides, acaricides, nematicides, fungicides, growth regulators or herbicides). The insecticides include, for example, carbamates, carboxylic esters, chlorinated hydrocarbons, phenylureas, substances produced by microorganisms, etc.

Examples of particularly favourable co-components include the following

### Fungicides:

### Inhibitors of Nucleic Acid Synthesis

benalaxyl, benalaxyl-M, bupirimate, chiralaxyl, clozylacon, dimethirimol, ethirimol, furalaxyl, hymexazol, metalaxyl, metalaxyl-M, ofurace, oxadixyl, oxolinic acid

### Inhibitors of Mitosis and Cell Division

benomyl, carbendazim, diethofencarb, fuberidazole, pencycuron, thiabendazole, thiophanatmethyl, zoxamide

### Inhibitors of Respiratory Chain Complex I

diflumetorim

### Inhibitors of Respiratory Chain Complex II

boscalid, carboxin, fenfuram, flutolanil, furametpyr, mepronil, oxycarboxin, penthiopyrad, thifluzamide

### Inhibitors of Respiratory Chain Complex III

azoxystrobin, cyazofamid, dimoxystrobin, enestrobin, famoxadone, fenamidone, fluoxastrobin, kresoxim-methyl, metominostrobin, orysastrobin, pyraclostrobin, picoxystrobin Decouplers dinocap, fluazinam

### Inhibitors of ATP Production

Fentin acetate, fentin chloride, fentin hydroxide, silthiofam

### Inhibitors of Amino Acid Biosynthesis and Protein Biosynthesis

andoprim, blasticidin-S, cyprodinil, kasugamycin, kasugamycin hydrochloride hydrate, mepanipyrim, pyrimethanil

### Inhibitors of Signal Transduction

fenpiclonil, fludioxonil, quinoxyfen

### Inhibitors of Lipid and Membrane Synthesis

chlozolinate, iprodione, procymidone, vinclozolin
ampropylfos, potassium-ampropylfos, edifenphos, iprobenfos (IBP), isoprothiolane, pyrazophos
tolclofos-methyl, biphenyl
iodocarb, propamocarb, propamocarb hydrochloride

### Inhibitors of Ergosterol Biosynthesis

fenhexamid,
azaconazole, bitertanol, bromuconazole, cyproconazole, diclobutrazole, difenoconazole, diniconazole, diniconazole-M, epoxiconazole, etaconazole, fenbuconazole, fluquinconazole, flusilazole, flutriafol, furconazole, furconazole-cis, hexaconazole, imibenconazole, ipconazole, metconazole, myclobutanil, paclobutrazole, penconazole, propiconazole, prothioconazole, simeconazole, tebuconazole, tetraconazole, triadimefon, triadimenol, triticonazole, uniconazole, voriconazole, imazalil, imazalil sulphate, oxpoconazole, fenarimol, flurprimidol, nuarimol, pyrifenox, triforine, pefurazoate, prochloraz, triflumizole, viniconazole,
aldimorph, dodemorph, dodemorph acetate, fenpropimorph, tridemorph, fenpropidin, spiroxamine,
naftifine, pyributicarb, terbinafine

### Inhibitors of Cell Wall Synthesis

benthiavalicarb, bialaphos, dimethomorph, flumorph, iprovalicarb, polyoxins, polyoxorim, validamycin A

### Inhibitors of Melanin Biosynthesis

Carpropamid, diclocymet, fenoxanil, phthalide, pyroquilon, tricyclazole

### Resistance Induction

acibenzolar-S-methyl, probenazole, tiadinil

### Multisite

captafol, captan, chlorothalonil, copper salts such as: copper hydroxide, copper naphthenate, copper oxychloride, copper sulphate, copper oxide, oxine-copper and Bordeaux mixture, dichlofluanid, dithianon, dodine, dodine free base, ferbam, fluorofolpet, guazatine, guazatine acetate, iminoctadine, iminoctadine albesilate, iminoctadine triacetate, mancopper, mancozeb, maneb, metiram, metiram zinc, propineb, sulphur and sulphur preparations containing calcium polysulphide, thiram, tolylfluanid, zineb, ziram

### Unknown Mechanism

amibromdol, benthiazol, bethoxazin, capsimycin, carvone, chinomethionat, chloropicrin, cufraneb, cyflufenamid, cymoxanil, dazomet, debacarb, diclomezine, dichlorophen, dicloran, difenzoquat, difenzoquat methyl sulphate, diphenylamine, ethaboxam, ferimzone, flumetover, flusulphamide, fluopicolide, fluoroimide, hexachlorobenzene, 8-hydroxyquinoline sulphate, irumamycin, methasulphocarb, metrafenone, methyl isothiocyanate, mildiomycin, natamycin, nickel dimethyl dithiocarbamate, nitrothal-isopropyl, octhilinone, oxamocarb, oxyfenthiin, pentachlorophenol and salts, 2-phenylphenol and salts, piperalin, propanosine-sodium, proquinazid, pyrrol nitrin, quintozene, tecloftalam, tecnazene, triazoxide, trichlamide, zarilamid and 2,3,5,6-tetrachloro-4-(methylsulphonyl)pyridine, N-(4-chloro-2-nitrophenyl)-N-ethyl-4-methylbenzenesulphonamide, 2-amino-4-methyl-N-phenyl-5-thiazolecarboxamide, 2-chloro-N-(2,3-dihydro-1,1,3-trimethyl-1H-inden-4-yl)-3-pyridine-carboxamide, 3-[5-(4-chlorophenyl)-2,3-dimethylisoxazolidin-3-yl]pyridine, cis-1-(4-chlorophenyl)-2-(1H-1,2,4-triazol-1-yl)cycloheptanol, 2,4-dihydro-5-methoxy-2-methyl-4-[[[[1-[3-(trifluoromethyl)phenyl]ethylidene]amino]oxy]methyl]phenyl]-3H-1,2,3-triazol-3-one (185336-79-2), methyl 1-(2,3-dihydro-2,2-dimethyl-1H-inden-1-yl)-1H-imidazole-5-carboxylate, 3,4,5-trichloro-2,6-pyridinedicarbonitrile, methyl 2-[[[cyclopropyl[(4-methoxyphenyl)imino]methyl]thio]methyl]-.alpha.-(methoxymethylene)benzacetate, 4-chloro-alpha-propynyloxy-N-[2-[3-methoxy-4-(2-propynyloxy)phenyl]ethyl]benzacetamide, (2S)—N-[2-[4-[[3-(4-chlorophenyl)-2-propynyl]oxy]-3-methoxyphenyl]ethyl]-3-methyl-2-[(methylsulphon-yl)amino]butanamide, 5-chloro-7-(4-methylpiperidin-1-yl)-6-(2,4,6-trifluorophenyl)[1,2,4]-triazolo[1,5-a]pyrimidine, 5-chloro-6-(2,4,6-trifluorophenyl)-N-[(1R)-1,2,2-trimethylpropyl]-[1,2,4]triazolo[1,5-a]pyrimidin-7-amine, 5-chloro-N-[(1R)-1,2-dimethylpropyl]-6-(2,4,6-trifluorophenyl)[1,2,4]triazolo[1,5-a]pyrimidin-7-amine, N-[1-(5-bromo-3-chloropyridin-2-yl)ethyl]-2,4-dichloronicotinamide, N-(5-bromo-3-chloropyridin-2-yl)methyl-2,4-dichloronicotinamide, 2-butoxy-6-iodo-3-propylbenzopyranon-4-one, N-{(Z)-[(cyclopropylmethoxy)-imino][6-(difluoromethoxy)-2,3-difluorophenyl]methyl}-2-benzacetamide, N-(3-ethyl-3,5,5-trimethylcyclohexyl)-3-formylamino-2-hydroxybenzamide, 2-[[[[1-[3(1-fluoro-2-phenylethyl)oxy]phenyl]ethylidene]amino]oxy]methyl]-alpha-(methoxyimino)-N-methyl-alpha-benzacetamide, N-{2-[3-chloro-5-(trifluoromethyl)pyridin-2-yl]ethyl}-2-(trifluoromethyl)benzamide, N-(3′,4′-dichloro-5-fluorobiphenyl-2-yl)-3-(difluoromethyl)-1-methyl-1H-pyrazole-4-carboxamide, N-(6-methoxy-3-pyridinyl)cyclopropanecarboxamide, 1-[(4-methoxyphenoxy)methyl]-2,2-dimethylpropyl-1H-imidazole-1-carboxylic acid, O-[1-[(4-methoxyphenoxy)methyl]-2,2-dimethylpropyl]-1H-imidazole-1-carbothioic acid, 2-(2-{[6-(3-chloro-2-methylphenoxy)-5-fluoropyrimidin-4-yl]oxy}phenyl)-2-(methoxyimino)-N-methylacetamide

### Bactericides:

bronopol, dichlorophen, nitrapyrin, nickel dimethyldithiocarbamate, kasugamycin, octhilinone, furancarboxylic acid, oxytetracycline, probenazole, streptomycin, tecloftalam, copper sulphate and other copper preparations.

### Insecticides/Acaricides/Nematicides:

### Acetylcholine Esterase (AChE) Inhibitors

Carbamates,
for example alanycarb, aldicarb, aldoxycarb, allyxycarb, aminocarb, bendiocarb, benfuracarb, bufencarb, butacarb, butocarboxim, butoxycarboxim, carbaryl, carbofuran, carbosulphan, cloethocarb, dimetilan, ethiofencarb, fenobucarb, fenothiocarb, formetanate, furathiocarb, isoprocarb, metam-sodium, methiocarb, methomyl, metolcarb, oxamyl, pirimicarb, promecarb, propoxur, thiodicarb, thiofanox, trimethacarb, XMC, xylylcarb triazamate
Organophosphates,
for example acephate, azamethiphos, azinphos (-methyl, -ethyl), bromophos-ethyl, bromfenvinfos (-methyl), butathiofos, cadusafos, carbophenothion, chlorethoxyfos, chlorfenvinphos, chlormephos, chlorpyrifos (-methyl/-ethyl), coumaphos, cyanofenphos, cyanophos, chlorfenvinphos, demeton-S-methyl, demeton-S-methylsulphon, dialifos, diazinon, dichlofenthion, dichlorvos/DDVP, dicrotophos, dimethoate, dimethylvinphos, dioxabenzofos, disulphoton, EPN, ethion, ethoprophos, etrimfos, famphur, fenamiphos, fenitrothion, fensulphothion, fenthion, flupyrazofos, fonofos, formothion, fosmethilan, fosthiazate, heptenophos, iodofenphos, iprobenfos, isazofos, isofenphos, isopropyl O-salicylate, isoxathion, malathion, mecarbam, methacrifos, methamidophos, methidathion, mevinphos, monocrotophos, naled, omethoate, oxydemeton-methyl, parathion (-methyl/-ethyl), phenthoate, phorate, phosalone, phosmet, phosphamidon, phosphocarb, phoxim, pirimiphos (-methyl/-ethyl), profenofos, propaphos, propetamphos, prothiofos, prothoate, pyraclofos, pyridaphenthion, pyridathion, quinalphos, sebufos, sulphotep, sulprofos, tebupirimfos, temephos, terbufos, tetrachlorvinphos, thiometon, triazophos, triclorfon, vamidothion

### Sodium Channel Modulators/Voltage-Dependent Sodium Channel Blockers

DDT
Oxadiazines,
for example indoxacarb

### Acetylcholine Receptor Agonists/Antagonists

Chloronicotinyls,
for example acetamiprid, clothianidin, dinotefuran, imidacloprid, nitenpyram, nithiazine, thiacloprid, thiamethoxam
Nicotine, bensultap, cartap

### Acetylcholine Receptor Modulators

Spinosyns,
for example spinosad

### GABA-Controlled Chloride Channel Antagonists

Organochlorines,
for example camphechlor, chlordane, endosulphan, gamma-HCH, HCH, heptachlor, lindane, methoxychlor
Fiprols,
for example acetoprole, ethiprole, fipronil, pyrafluprole, pyriprole, vaniliprole

### Chloride Channel Activators

Mectins,
for example avermectin, emamectin, emamectin-benzoate, ivermectin, milbemycin

### Juvenile Hormone Mimetics,

for example diofenolan, epofenonane, fenoxycarb, hydroprene, kinoprene, methoprene, pyriproxifen, triprene

### Ecdysone Agonists/Disruptors

Diacylhydrazines,
for example chromafenozide, halofenozide, methoxyfenozide, tebufenozide

### Chitin Biosynthesis Inhibitors

Benzoylureas,
for example bistrifluoron, chlofluazuron, diflubenzuron, fluazuron, flucycloxuron, flufenoxuron, hexaflumuron, lufenuron, novaluron, noviflumuron, penfluoron, teflubenzuron, triflumuron
Buprofezin
Cyromazine

### Oxidative Phosphorylation Inhibitors, ATP Disruptors

Diafenthiuron
Organotin compounds,
for example azocyclotin, cyhexatin, fenbutatin-oxide

### Oxidative Phosphorylation Decouplers Acting by Interrupting the H-Proton Gradient

Pyrroles,
for example chlorfenapyr
Dinitrophenols,
for example binapacyrl, dinobuton, dinocap, DNOC

### Site-I Electron Transport Inhibitors

METI's,
for example fenazaquin, fenpyroximate, pyrimidifen, pyridaben, tebufenpyrad, tolfenpyrad
Hydramethylnon
Dicofol

### Site-II Electron Transport Inhibitors

Rotenone

### Site-III Electron Transport Inhibitors

Acequinocyl, fluacrypyrim

### Microbial Disruptors of the Insect Gut Membrane

Bacillus thuringiensis strains

### Lipid Synthesis Inhibitors

Tetronic acids,
for example spirodiclofen, spiromesifen
Tetramic acids,
for example spirotetramate (CAS Reg. No.: 203313-25-1) and 3-(2,5-dimethylphenyl)-8-methoxy-2-oxo-1-azaspiro[4.5]dec-3-en-4-yl ethyl carbonate (alias: carbonic acid, 3-(2,5-dimethylphenyl)-8-methoxy-2-oxo-1-azaspiro[4.5]dec-3-en-4-yl ethyl ester, CAS Reg. No.: 382608-10-8)
Carboxamides,
for example flonicamid
Octopaminergic agonists,
for example amitraz

### Inhibitors of Magnesium-Stimulated ATPase,

Propargite
Benzodicarboxamides,
for example flubendiamide
Nereistoxin analogues,
for example thiocyclam hydrogen oxalate, thiosultap-sodium

### Biologicals, Hormones or Pheromones,

azadirachtin, Bacillus spec., Beauveria spec., codlemone, Metarrhizium spec., Paecilomyces spec., thuringiensin, Verticillium spec.
   Active Compounds with Unknown or Unspecific Mechanisms of Action

### Fumigants,

for example aluminium phosphide, methyl bromide, sulphuryl fluoride

### Antifeedants,

for example cryolite, flonicamid, pymetrozine

### Mite Growth Inhibitors,

for example clofentezine, etoxazole, hexythiazox
amidoflumet, benclothiaz, benzoximate, bifenazate, bromopropylate, buprofezin, quinomethionate, chlordimeform, chlorobenzilate, chloropicrin, clothiazoben, cycloprene, cyflumetofen, dicyclanil, fenoxacrim, fentrifanil, flubenzimine, flufenerim, flutenzin, gossyplure, hydramethylnone, japonilure, metoxadiazone, petroleum, piperonyl butoxide, potassium oleate, pyridalyl, sulphluramid, tetradifon, tetrasul, triarathene, verbutin

The amount of the individual components can be varied within a relatively wide range in the oil-based suspension concentrates of the invention. Thus the concentrations
of active agrochemicals are between 5% and 40%, preferably between 10% and 37.5%, very preferably between 12.5% and 35% by weight,
of penetrant are between 5% and 55%, preferably between 10% and 35% by weight,
of vegetable oil are between 15% and 55%, preferably between 20% and 50% by weight,
of cyclohexanone are between 5% and 20%, preferably between 7% and 16% by weight,
of surfactants and/or dispersants are between 2.5% and 30%, preferably between 5.0% and 25% by weight, and
of additives are between 0.1% and 25%, preferably between 0.1% and 20% by weight.

The oil-based suspension concentrates of the invention are produced by mixing the components with one another in the respectively desired proportions. The order in which the constituents are combined with one another is arbitrary. Appropriately the solid components are used in a finely ground state. It is, however, also possible to subject the suspension which results after the constituents have been combined first to a coarse grinding and then to a fine grinding, so that the mean particle size is below 20 μm. Preferred suspension concentrates are those in which the solid particles have a mean size between 1 and 10 μm.

The temperatures when carrying out the process of the invention can be varied within a certain range. The work is carried out generally at temperatures between 10° C. and 60° C., preferably between 15° C. and 40° C.

Equipment suitable for carrying out the process of the invention includes customary mixing and grinding apparatus which is used for producing agrochemical formulations.

The oil-based suspension concentrates of the invention constitute formulations which remain stable even following prolonged storage at elevated temperatures or in the cold, since no significant crystal growth is observed. By dilution with water they can be converted into homogeneous spray liquids. These spray liquids are applied by customary methods, i.e., for example, by spraying, pouring or injecting.

The application rate of the oil-based suspension concentrates of the invention can be varied within a relatively wide range. It is guided by the particular active agrochemicals and by their amount in the formulations.

With the aid of the oil-based suspension concentrates of the invention it is possible to deliver active agrochemicals particularly from the class of the neonicotinoids, to plants and/or their habitat in a particularly advantageous way.

With the formulations of the invention it is possible to treat all plants and plant parts. By plants here are meant all plants and plant populations, such as desirable and unwanted wild plants or crop plants (including naturally occurring crop plants). Crop plants may be plants which can be obtained by conventional breeding and optimization methods or by biotechnological and gene-technological methods or combinations of these methods, including the transgenic plants and including the plant cultivars which can or cannot be protected by varietal property rights. By plant parts are to be meant all above-ground and below-ground parts and organs of the plants, such as shoot, leaf, flower and root, an exemplary listing embracing leaves, needles, stems, trunks, flowers, fruit bodies, fruits and seeds and also roots, tubers and rhizomes. The plant parts also include harvested material and also vegetative and generative propagation material, examples being seedlings, tubers, rhizomes, cuttings and seeds.

What may be emphasized in this context is the particularly advantageous effect of the compositions according to the invention with regard to their use in cereal plants such as, for example, wheat, oats, barley, spelt, triticale and rye, but also in maize, sorghum and millet, rice, sugar cane, soya beans, sunflowers, potatoes, cotton, oilseed rape, canola, tobacco, sugar beet, fodder beet, asparagus, hops and fruit plants (comprising pome fruit such as, for example, apples and pears, stone fruit such as, for example, peaches, nectarines, cherries, plums and apricots, citrus fruits such as, for example, oranges, grapefruits, limes, lemons, kumquats, tangerines and satsumas, nuts such as, for example, pistachios, almonds, walnuts and pecan nuts, tropical fruits such as, for example, mango, papaya, pineapple, dates and bananas, and grapes) and vegetables (comprising leaf vegetables such as, for example, endives, corn salad, Florence fennel, lettuce, cos lettuce, Swiss chard, spinach and chicory for salad use, cabbages such as, for example, cauliflower, broccoli, Chinese leaves, Brassica oleracea (L.) convar. acephala var. sabellica L. (curly kale, feathered cabbage), kohlrabi, Brussels sprouts, red cabbage, white cabbage and Savoy cabbage, fruit vegetables such as, for example, aubergines, cucumbers, capsicums, table pumpkins, tomatoes, courgettes and sweetcorn, root vegetables such as, for example celeriac, wild turnips, carrots, including yellow cultivars, Raphanus sativus var. niger and var. radicula, beetroot, scorzonera and celery, legumes such as, for example, peas and beans, and vegetables from the Allium family such as, for example, leeks and onions).

The treatment of the plants and plant parts in accordance with the invention with the inventive formulations is carried out directly or by action on their environment, habitat or storage area in accordance with the customary treatment methods, for example by dipping, spraying, vaporizing, atomizing, broadcasting or painting on and, in the case of propagation material, especially seeds, additionally by single or multiple coating.

The active agrochemicals comprised develop a better biological activity than when applied in the form of the corresponding conventional formulations.

The invention is illustrated by the following examples.

### EXAMPLES

### Preparation Examples

### Example 1

To prepare a suspension concentrate

| | |
|---|---|
| 144.0 | G of imidacloprid |
| 38.4 | G of deltamethrine |
| 100.0 | G of Arlatone ® T |
| 75.0 | G of cyclohexanone |
| 130.0 | G of Atlox ® 3467 |
| 20.0 | G of lignosulphonate (Borresperse ® NA) |
| 25.0 | G of propylene glycol |
| 0.5 | G of polydimethylsiloxane |
| 2.0 | G of anhydrous citric acid |
| 2.0 | G of 2,6-di-tert-butyl-4-methylphenol |

are introduced with stirring at room temperature into a mixture of

| | |
|---|---|
| 200.0 | G of the compound of formula (I-c-1) and |
| 263.1 | G of sunflower oil |

After the end of the addition the mixture is stirred at room temperature for a further 10 minutes. The resultant homogeneous suspension is subjected first to coarse grinding and then to fine grinding, giving a suspension in which 90% of the solid particles have a size below 6 μm.

The formulas below were produced in the same way as for Example 1.

TABLE 1

| | 2 | 3 | 4 | 5 | 6 | 7 | 8 | 9 | 10 |
|---|---|---|---|---|---|---|---|---|---|
| Beta-Cyfluthrin | | 85.5 | | 46.5 | 84 | 84 | 84 | 85.5 | 84 |
| Deltamethrin | 38.5 | | 38.5 | | | | | | |
| Imidacloprid | 144 | 198 | 144 | 102 | 196 | 196 | 196 | 198 | 196 |
| Thiacloprid | | | | | | | | | |
| 2,6-Di-tert-butyl-4- | 2 | 2 | 2 | 2 | 2 | 2 | 2 | 2 | 2 |
| Methylphenol | | | | | | | | | |
| Agnique ® KE 3552 | | 150 | | | | | | | |
| Arlatone ® T | 100 | 100 | | 100 | 100 | 100 | 100 | 100 | 100 |
| Atlox ® 3467 | | | 113 | | | | | | |
| Atlox ® 4894 | 50 | 50 | | 50 | 80 | 50 | | 50 | |
| Atlox ® 4913 | | | | | | | | | |
| (anhydrous) | | | | | | | | | |
| Atlox ® 4914 | | | | | | | 50 | | 50 |
| Borresperse ® NA | | | 20 | | | | | | 5 |
| Cyclohexanone | 75 | 150 | 75 | 75 | 100 | 100 | 150 | 150 | 150 |
| Genagen ® 4166 | | | | | | | | | |
| Kraftsperse ® DW 5 | | | | | | 5 | | | |
| Maize oil | 383 | | | | | | | | |
| Morwet ® D 425 | 5 | 5 | | 5 | | | 5 | 5 | |
| N-Methylpyrrolidone | | | | | | | | | |
| Polydimethylsiloxane | 0.5 | 0.5 | 0.5 | 0.5 | 0.5 | 0.5 | 0.5 | 0.5 | 0.5 |
| Propylene glycol | | | 25 | | | | | | |
| Solvesso ® 100 | | | | | | | | | |
| Sunflower oil | | 257 | 280 | 417 | 235.5 | 260.5 | 260.5 | 257 | 260.5 |
| Trylox ® 6746 | | | 100 | | | | | | |
| Compound (I-c-1) | 200 | | 200 | 200 | 200 | 200 | 150 | | 150 |
| Compound (I-d-1) | | | | | | | | | |
| Compound (I-f-1) | | | | | | | | 150 | |
| Anhydrous citric acid | 2 | 2 | 2 | 2 | 2 | 2 | 2 | 2 | 2 |

| | 11 | 12 | 13 | 14 | 15 | 16 | 17 | 18 | 19 | 20 |
|---|---|---|---|---|---|---|---|---|---|---|
| Beta-Cyfluthrin | | 46.5 | 46.5 | 85.5 | | 85.5 | | | | 85.5 |
| Deltamethrin | 38.5 | | | | 38.5 | | 38.5 | 38.5 | 72.5 | |
| Imidacloprid | 144 | 102 | 102 | 198 | 144 | 198 | 144 | 144 | | 198 |
| Thiacloprid | | | | | | | | | 143 | |
| 2,6-Di-tert-butyl-4- | 2 | 2 | 2 | 2 | 2 | 2 | 2 | 2 | 2 | 2 |
| Methylphenol | | | | | | | | | | |
| Agnique ® KE 3552 | 200 | | | | | | | | | |
| Arlatone ® T | 100 | 100 | 100 | | 100 | 100 | | 100 | 100 | 100 |
| Atlox ® 3467 | | 113 | | | 130 | | 130 | | 110 | |
| Atlox ® 4894 | 50 | | | 50 | | 50 | | 50 | | 50 |
| Atlox ® 4913 | | | | | 30 | | | | | |
| (anhydrous) | | | | | | | | | | |
| Atlox ® 4914 | | | 50 | | | | | | | |
| Borresperse ® NA | | | | | 30 | | | | | |
| Cyclohexanone | 75 | 75 | 75 | 150 | 75 | 150 | 100 | 75 | 125 | 150 |
| Genagen ® 4166 | | | | | | | | | | |
| Kraftsperse ® DW 5 | | | | | | | | | | |
| Maize oil | | | 417 | | | | | | | |
| Morwet ® D 425 | 5 | | 5 | 5 | | 5 | | 5 | | 5 |
| N-Methylpyrrolidone | | | | | | | | | | |
| Polydimethylsiloxane | 0.5 | 0.5 | 0.5 | 0.5 | 0.5 | 0.5 | 0.5 | 0.5 | 0.5 | 0.5 |
| Propylene glycol | | | | | 25 | | | | | |
| Solvesso ® 100 | | | | | | | | | | |
| Sunflower oil | 383 | 359 | | 257 | 223 | 257 | 283 | 383 | 245 | 257 |
| Trylox ® 6746 | | | | 100 | | | 100 | | | |
| Compound (I-c-1) | | 200 | 200 | 150 | 200 | 150 | 200 | | 200 | |
| Compound (I-d-1) | | | | | | | | 200 | | 150 |
| Compound (I-f-1) | | | | | | | | | | |
| Anhydrous citric acid | 2 | 2 | 2 | 2 | 2 | 2 | 2 | 2 | 2 | 2 |

| | 21 | 22 | 23 | 24 | 25 |
|---|---|---|---|---|---|
| Beta-Cyfluthrin | | | 84 | 46.5 | 85.5 |
| Deltamethrin | 72.5 | 38.5 | | | |
| Imidacloprid | | 144 | 196 | 102 | 198 |
| Thiacloprid | 143 | | | | |
| 2,6-Di-tert-butyl-4-methylphenol | 2 | 2 | 2 | 2 | 2 |
| Agnique ® KE 3552 | | | | | |
| Arlatone ® T | 100 | 100 | 75 | 100 | 100 |
| Atlox ® 3467 | | | | 113 | |
| Atlox ® 4894 | 50 | 50 | | | 50 |
| Atlox ® 4913 (anhydrous) | | | | | |
| Atlox ® 4914 | | | 50 | | |
| Borresperse ® NA | | | 20 | | |
| Cyclohexanone | 125 | 75 | 200 | 75 | 150 |
| Genagen ® 4166 | | | | | |
| Kraftsperse ® DW 5 | | | | | |
| Maize oil | | | | | |
| Morwet ® D 425 | 5 | 5 | | | 5 |
| N-Methylpyrrolidone | | | | | |
| Polydimethylsiloxane | 0.5 | 0.5 | 0.5 | 0.5 | 0.5 |
| Propylene glycol | | | | | |
| Solvesso ® 100 | | | | | |
| Sunflower oil | 300 | 383 | 220.5 | 359 | 257 |
| Trylox ® 6746 | | | | | |
| Compound (I-c-1) | 200 | 200 | 150 | | |
| Compound (I-d-1) | | | | 200 | |
| Compound (I-f-1-1) | | | | | 150 |
| Anhydrous citric acid | 2 | 2 | 2 | 2 | 2 |

### Comparative Examples

The formulas below were produced in the same way as for Example 1.

TABLE 2

| | 1 | 2 | 3 | 4 | 5 | 6 | 7 |
|---|---|---|---|---|---|---|---|
| Beta-Cyfluthrin | | | 94.5 | 46.5 | 85.5 | 85.5 | 85.5 |
| Deltamethrin | 39.5 | 94.5 | | | | | |
| Imidacloprid | 147 | 187 | 187 | 102 | 198 | 198 | 198 |
| Thiacloprid | | | | | | | |
| 2,6-Di-tert-butyl-4- | 2 | 2 | 2 | 2 | 2 | 2 | 2 |
| methylphenol | | | | | | | |
| Agnique ® KE 3552 | | | | | | | |
| Arlatone ® T | 100 | 100 | 100 | 100 | 100 | 100 | 100 |
| Atlox ® 3467 | 130 | 130 | 130 | 113 | | | |
| Atlox ® 4894 | | | | | 50 | 50 | 50 |
| Atlox ® 4913 | | | | | | | |
| (anhydrous) | | | | | | | |
| Atlox ® 4914 | | | | | | | |
| Borresperse ® NA | | 20 | | | | | |
| Cyclohexanone | | | | | | | |
| Genagen ® 4166 | | | | | | | 150 |
| Kraftsperse ® DW 5 | | | | | | | |
| Maize oil | | | | | | | |
| Morwet ® D 425 | | | 20 | | 5 | 5 | 5 |
| N-Methylpyrrolidone | | | | | | 150 | |
| Polydimethylsiloxane | 0.5 | 0.5 | 0.5 | 0.5 | 0.5 | 0.5 | 0.5 |
| Propylene glycol | | | | | | | |
| Solvesso ® 100 | | | | 75 | 150 | | |
| Sunflower oil | 379 | 264 | 264 | 359 | 257 | 257 | 257 |
| Trylox ® 6746 | | | | | | | |
| Compound (I-c-1) | 200 | 200 | 200 | 200 | 150 | 150 | 150 |
| Compound (I-d-1) | | | | | | | |
| Compound (I-f-1) | | | | | | | |
| Anhydrous citric acid | 2 | 2 | 2 | 2 | 2 | 2 | 2 |

The components in the compositions of the invention that are defined by means of their trade names are available from the following suppliers:

| Trade name | Type of compound | Supplier |
|---|---|---|
| Agnique ® KE 3552 | alkanol alkoxylate | Cognis |
| Arlatone ® T | PEG-40 sorbitan peroleate, | Uniqema |
| | nonionic | |
| Atlox ® 3467 | blend containing alkylaryl | Uniqema |
| | sulphonate, ethylhexanol, | |
| | ethoxylated alcohol | |
| Atlox ® 4894 | polyalkoxylated alcohol | Uniqema |
| Atlox ® 4913 | polymeric nonionic surfactant | Uniqema |
| (anhydrous) | | |
| Atlox ® 4914 | polymeric nonionic surfactant | Uniqema |
| Borresperse ® NA | lignosulphonate | Borregaard |
| | | LignoTech |
| Genagen ® 4166 | caprylic/capric fatty acid | Clariant |
| | dimethylamide | |
| Kraftsperse ® DW 5 | lignosulphonate, sodium salt | Westvaco |
| Morwet ® D 425 | naphthalene sulphonate | Witco |
| Solvesso ® 100 | aromatic organic solvent | Exxon Mobile |
| Trylox ® 6746 | PEG-40 sorbitol hexaoleate | Cognis |

### Crystallization Behaviour

The crystallization behaviour is investigated by storing 100 ml of formulation for eight weeks under fluctuating temperature conditions. The temperature conditions are as follows:
48 hours at 30° C.,
reduction in temperature over 22.5 hours at 2° C./hour down to −15° C.,
75 hours at −15° C.,
increase in temperature over 22.5 hours at 2° C./hour up to 30° C.

Following storage, the sample is brought to room temperature and the crystallization behaviour is examined.

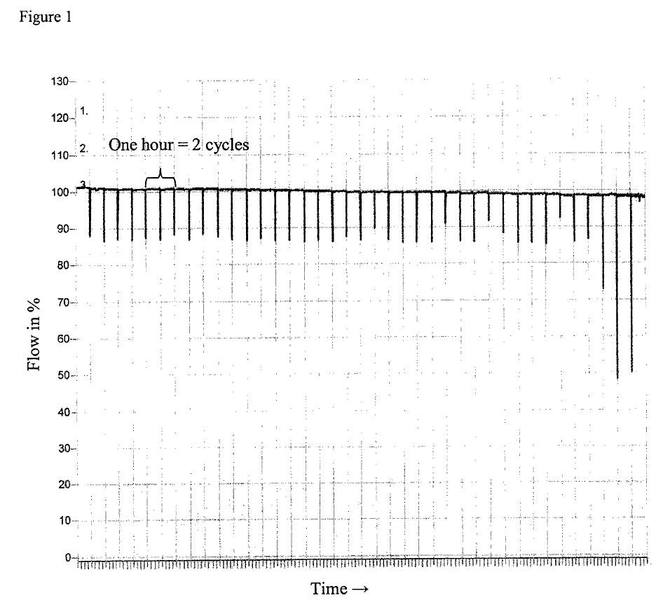

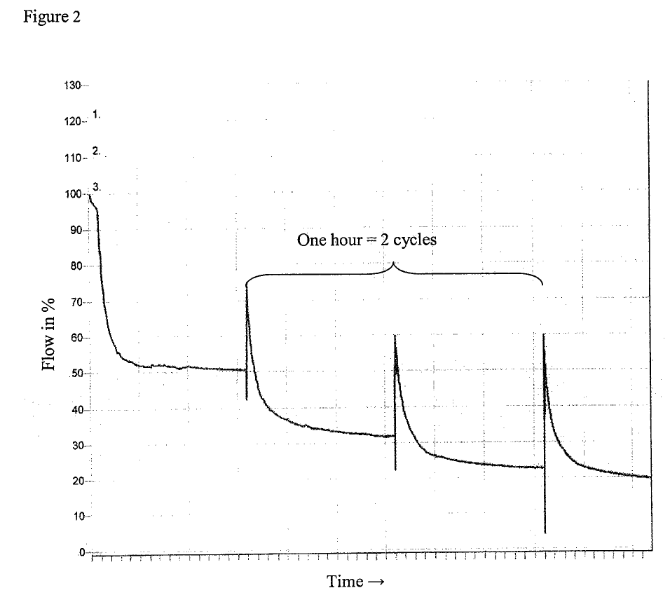

The crystallization properties are tested by pumping 500 ml in each case of an aqueous spray liquor with a concentrate content of 0.5% by weight in circulation in a flow-traversed apparatus, by means of a pump, through a fine-meshed screen for 30 minutes. In the course of this procedure the flow over the screen is measured. At the same level of flow, forty repetitions of this operation are carried out, with 500 ml of freshly employed spray liquor in each case. Crystal growth in the formulations tested will lead to blocking of the screen and so will cause a loss of flow over the screen. If the flow is below 20%, the measurement cycle is discontinued. By way of example, 2 results are reproduced as graphs. Graph 1 shows the result of a flow test with an inventive formulation, for which the flow is still unchanged after forty cycles (20 hours). Graph 2 shows the result for a comparative formula. After four cycles (2 hours) the flow has dropped to 20% (see FIGS. 1 and 2).

FIG. 1: result of a flow test with inventive formulation 16, measured over 40 cycles

FIG. 2: result of a flow test with comparative formulation 3, measured over 4 cycles

### Use Example II

### Crystallization Behaviour

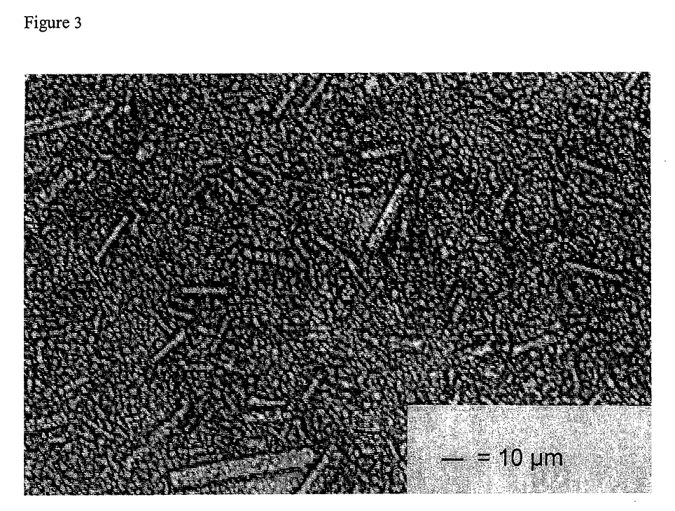

After eight weeks of storage of the formulation under fluctuating temperature conditions at 54° C. the growth of the active substance crystals is determined by means of light microscopy. Immediately after production, all formulations exhibit particle sizes of up to 10 micrometres. All of the inventive formulations exhibit particle sizes after storage of up to a maximum of 20 micrometres. The comparative formulations exhibit substantially coarser particles, up to more than 100 micrometres (see FIGS. 3 to 5).

FIG. 3: light-microscope investigation of Comparative Example 3 after above-described eight-week storage

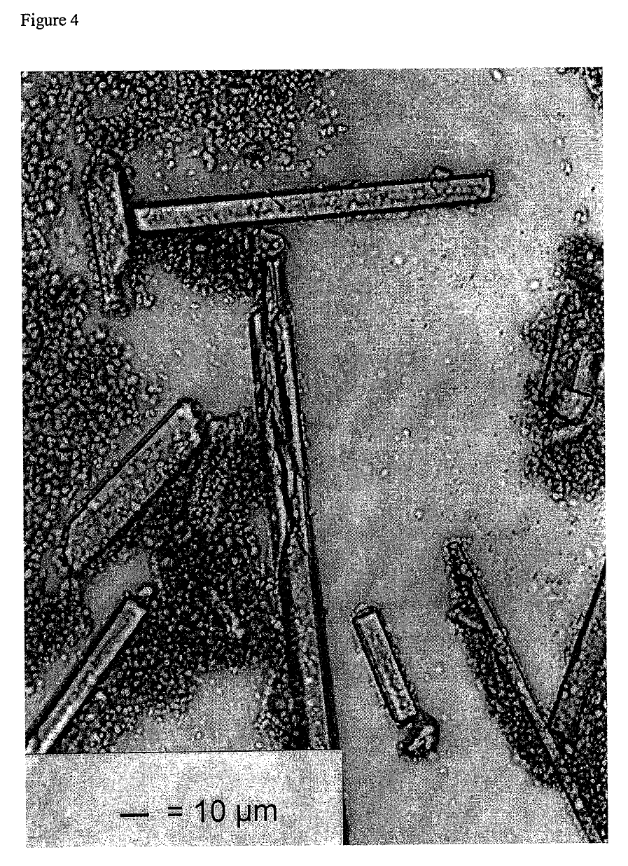

FIG. 4: light-microscope investigation of Comparative Example 1 after above-described eight-week storage

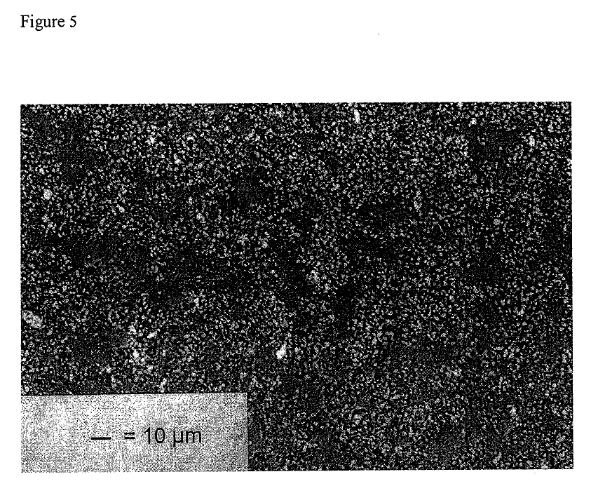

FIG. 5: light-microscope investigation of inventive formulation 16 after above-described eight-week storage

### Examples of Biological Action

### Knock-Down Action

### Myzus persicae Test

An appropriate application solution is produced by diluting 1 part by weight of formulated product with water to the desired concentration.

Pepper plants (Capsicum annuum) infested by all stages of the green peach aphid (Myzus persicae) are sprayed with an application solution at the desired concentration.

Immediately after the spray coating has dried off the action is measured in %. 100% means that all of the aphids have been damaged; 0% means that no aphids have been damaged.

In this test the following formulations, for example, exhibit superior activity over the prior art: 15, 16.

TABLE 3
Phytopathogenic insects
Myzus persicae test

| | Concentration | Kill |
|---|---|---|
| Active substance/Product | in g ai/ha | in % after 2 ^{h} |
| Example 16 | 1 + 0.43 | 80 |
| inventive | | |
| Example 15 | 1 + 0.27 | 90 |
| inventive | | |
| Imidacloprid OD 200 | 1 | 60 |
| prior art | | |
| β-Cyfluthrin EC 100 | 0.43 | 50 |
| prior art | | |
| β-Cyfluthrin SC 125 | 0.43 | 20 |
| prior art | | |
| Deltamethrin EC 025 | 0.27 | 80 |
| prior art | | |
| Deltamethrin SC 200 | 0.27 | 0 |
| prior art | | |

Mortality/Efficacy: Myzus persicae Test

An appropriate application solution is produced by diluting 1 part by weight of formulated product with water to the desired concentration.

Pepper plants (Capsicum annuum) infested by all stages of the green peach aphid (Myzus persicae) are sprayed with an application solution at the desired concentration.

After the desired time the action is measured in %. 100% means that all of the aphids have been killed; 0% means that no aphids have been killed.

In this test the following formulations, for example, exhibit superior activity over the prior art: 15, 16.

TABLE 4
Phytopathogenic insects
Myzus persicae test

| | Concentration | Kill |
|---|---|---|
| Active substance/Product | in g ai/ha | in % after 1 d |
| Example 16 | 1 + 0.43 | 100 |
| inventive | | |
| Example 15 | 1 + 0.27 | 98 |
| inventive | | |
| Imidacloprid OD 200 | 1 | 94 |
| prior art | | |
| β-Cyfluthrin EC 100 | 0.43 | 55 |
| prior art | | |
| β-Cyfluthrin SC 125 | 0.43 | 20 |
| prior art | | |
| Deltamethrin EC 025 | 0.27 | 94 |
| prior art | | |
| Deltamethrin SC 200 | 0.27 | 0 |
| prior art | | |

### Test Description: Penetrants at the Level of the Cuticle

Additives which act as penetrants at the level of the cuticle may be referred to below as accelerator additives (cf. Schönherr and Baur, 1994, Pesticide Science 42, 185-208). The characterizing feature of accelerator additives is their ability to penetrate from the aqueous spray liquor and/or from the spray coating into the cuticle and thereby to increase the mobility of active substances in the cuticle. Other additives such as polyethylene glycol, in contrast, act only in the spray coating (via the liquid phase) or act only as wetting agents, such as sodium dodecyl sulphate, for example.

This test determines the influence of additives on the penetration properties of other substances at the level of the cuticle. The mobility of a test substance in the cuticle is measured with and without an additive, via a desorption method. The method is published in detail in the literature (Baur et al., 1997, Pesticide Science, 51, 131-152), and only the principles and deviations are described below.

As a test substance with the function of a tracer a selection was made here of a radiolabelled weak organic acid. Plant material used comprised the enzymatically isolated leaf cuticles of the top face of peach leaves from outdoor trees. The cuticles were installed in specially manufactured stainless steel diffusion cells. The tracer, in a citrate buffer at a pH of 3 in the dissolved state, was applied to the side originally facing the inside of the leaf. This inner side readily takes up the small radioactive amount of the tracer in the undissociated acid form. Subsequently this inner side was covered and maintained at 100% atmospheric humidity. The morphological outer side of the leaf cuticle, normally exposed to the air, was then contacted with a buffer (pH 7), with the receptor solution, and desorption was started. The penetrated acid form of the test substance is dissociated by the receptor and the desorption follows first-order kinetics. The desorption constant is proportional to the mobility of the tracer in the cuticle.

After at least 2 times for determining this constant, desorption is then continued with a buffer which additionally includes the test additive. Depending on the property of the additive there is then sorption of the additive in the cuticle and, depending on its activity as a plasticizer for the cuticle, there is an increase in the mobility of the tracer within the cuticle. This is manifested in an increased desorption constant, and the ratio of the slopes with additive to the slope without additive describes the effect of the additive to act as a penetrant at the level of the cuticle. The comparison of the average effect of different additives shows their effectiveness to act as cuticle plasticizers.

1. A composition comprising
at least one room-temperature-solid active substance from the class of the neonicotinoids,
at least one room-temperature-solid active substance from the class of the pyrethroids,
at least one penetrant,
at least one vegetable oil,
cyclohexanone,
at least one nonionic surfactant and/or at least one anionic surfactant, and
one or more additives selected from the group consisting of emulsifiers, foam inhibitors, preservatives, antioxidants, spreaders, colorants and a thickener or combinations thereof.

2. The composition according to claim 1, wherein the neonicotinoid is selected from the group consisting of thiamethoxam, clothianidin, thiacloprid, dinetofuran, acetamiprid, nitenpyram and imidacloprid.

3. The composition according to claim 2, wherein the neonicotinoid is imidacloprid.

4. The composition according to claim 1, wherein the pyrethroid is beta-cyfluthrin or deltamethrin.

5. The composition according to claim 1, wherein the penetrant is an alkanol alkoxylate of the formula
R—O—(-AO)ₘR′ (I)
in which
R is linear or branched alkyl having 4 to 20 carbon atoms;
R′ is H, methyl, ethyl, n-propyl, isopropyl, n-butyl, isobutyl, tert-butyl, n-pentyl or n-hexyl;
AO is an ethylene oxide radical, a propylene oxide radical, a butylene oxide radical or mixtures of ethylene oxide and propylene oxide radicals or butylene oxide radicals; and
m is a number from about 2 to 30.

6. The composition according to claim 5, wherein the penetrant is an alkanol alkoxylate of the formula
R—O—(-EO—)ₙ—R′ (I-a),
R—O—(-EO—)ₚ—(—PO—)_{q}—R′ (I-b),
R—O—(—PO—)ᵣ-(EO—)ₛ—R′ (I-c),
R—O—(-EO—)ₚ—(—BO—)_{q}—R′ (I-d),
R—O—(—BO—)ᵣ—(-EO—)ₛ—R′ (I-e), or
CH₃—(CH₂)ₜ—CH₂—O—(—CH₂—CH₂—O—)ᵤ—R′ (I-f)
in which
R and R′ are as defined in claim 5,
EO is —CH₂—CH₂—O—,
PO is 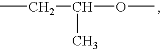
BO is 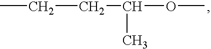
n is an integer from 2 to 20,
p, q, r and s independently are integers from 1 to 10,
t is an integer from 8 to 13, and
u is an integer from 6 to 17.

7. The composition according to claim 6, wherein the penetrant is an alkanol alkoxylate of the formula 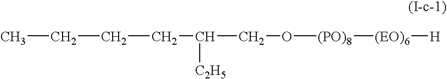
in which the numbers 8 and 6 are average values,
CH₃—(CH₂)₁₀—O—(-EO—)₆—(—BO—)₂—CH₃ (I-d-1)
in which the numbers 10, 6 and 2 are average values, or
CH₃—(CH₂)_{10.5}—CH₂—O—(—CH₂—CH₂—O—)_{8.4}—H (I-f-1)
in which the numbers 10.5 and 8.4 are average values.

8. The composition according to claim 6, wherein the penetrant is an alkanol alkoxylate of the formula
CH₃—(CH₂)_{10.5}—CH₂—O—(—CH₂—CH₂—O—)_{8.4}—H (I-f-1-1)
in which the numbers 10.5 and 8.4 are average values.

9. The composition according to claim 1 comprising
between 5% and 40% by weight of active agrochemicals,
between 5% and 55% by weight of penetrant,
between 15% and 55% by weight of vegetable oil,
between 5% and 20% by weight of cyclohexanone,
between 2.5% and 30% by weight of surfactants or dispersants or a combination thereof, and
between 0.1% and 25% by weight of additives.

10. The composition according to claim 9, comprising
between 10% and 37.5% by weight of active agrochemicals,
between 10% and 35% by weight of penetrant,
between 20% and 50% by weight of vegetable oil,
between 7% and 16% by weight of cyclohexanone,
between 5% and 25% by weight of surfactants and/or or dispersants or a combination thereof, and
between 0.1% and 20% by weight of additives.

11. A process for producing a composition according to claim 1 comprising mixing one or more compounds of the composition and grinding said compounds until an average particle size of said compounds is less than 10 μm.

12. The process according to claim 11, wherein said grinding comprises a coarse grinding and a fine grinding out until 90% of the particles of said compounds have a size of less than 6 μm.

13. The process according to claim 11, wherein said penetrant and said vegetable oil are added together initially followed by the addition of the remaining compounds of is said composition.

14. A method of controlling harmful insects, comprising contacting said insects or their habitat with an effective amount of an undiluted or diluted composition according to claim 1.
